(19) United States
(12) Patent Application Publication
(10) Pub. No.: US 2026/0294251 A1
(43) Pub. Date: Oct. 1, 2026

(54) Method for Medical Imaging

(71) Applicant: Integro Theranositcs, LLC, Chesterfield, MO (US)

(72) Inventors: Nick Staten, Saint Louis, MO (US); Daniel J. Thompson, Saint Louis, MO (US); Scott Glover, Chesterfield, MO (US); Jim Joffrion, Saint Louis, MO (US); Robert Birch, Chesterfield, MO (US); Clara C. Li, Chesterfield, MO (US); Peter Langecker, Chesterfield, MO (US)

(73) Assignee: Integro Theranositcs, LLC, Chesterfield, MO (US)

(21) Appl. No.: 19/479,594

(22) PCT Filed: May 8, 2024
(86) PCT No.: PCT/US2024/028386

§ 371 Date: Oct. 28, 2025

Related U.S. Application Data
(60) Provisional application No. 63/500,631, filed on May 8, 2023.

(51) Int. Cl. A61B 5/00; A61K 47/02; A61K 47/12; A61K 47/22; A61K 47/26; A61K 47/40; A61K 49/00; A61M 5/00

(52) U.S. Cl. CPC A61B 5/0071; A61B 5/0091; A61K 47/02; A61K 47/12; A61K 47/22; A61K 47/26; A61K 47/40; A61K 49/0032; A61K 49/0056; A61M 5/007

(57) ABSTRACT

Provided is a method for medical imaging in a patient in need thereof, comprising administering a pharmaceutical composition comprising LS301 to the patient via slow intravenous injection at a dose between about 0.001 mg/kg and about 0.5 mg/kg. Also provided is a method for surgical removal of cancer in a patient in need thereof, comprising administering a pharmaceutical composition comprising LS301 to the patient via slow intravenous injection at a dose between about 0.025 mg/kg and about 0.001 mg/kg before surgery for cancer resection; imaging via near-infrared fluorescence of the LS301 to identify malignant tissue in the patient, wherein each patient serves as their own control in terms of the near-infrared (NIR) light on versus the NIR light off; and surgically removing the image-identified malignant tissue from the patient, wherein the imaging step occurs during the surgical removal step.

This application claims the benefit of priority of U.S. Provisional Patent Application Ser. No. 63/500,631 filed May 8, 2023, the disclosure of which is incorporated by reference in its entirety for all purposes.

The present disclosure generally relates to medical imaging in a patient in need thereof using near-infrared fluorescence.

Initial management of a breast cancer diagnosis usually involves surgery to simultaneously remove the tumor and stage the disease. Breast surgery may consist of a lumpectomy (partial mastectomy) or a mastectomy. Lumpectomy is considered a “breast-conserving surgery” procedure associated with better cosmesis and represents the majority of procedures performed today. However, excessive tissue removal beyond the positive margin may result in poor cosmetic results.

The fear is incomplete tumor removal, which increases the chances of cancer recurrence and can result in further surgical intervention. In general, surgeons still rely on visual inspection, palpation, and tactile evaluation to distinguish cancer from noncancerous tissue intraoperatively, leading to subjective decision-making and variable outcomes. The current standard of care is to excise just enough tissue to achieve a negative margin because positive margins are associated with higher rates of local recurrence or possible metastatic spread.

Sentinel lymph node biopsy (SLNB) is the standard of care for staging breast cancer for certain diagnosis, such as invasive ductal carcinoma (IDC), the most common form. In addition, axillary lymph-node staging is typically performed simultaneously via SLNB. While standard imaging methods, such as mammography and ultrasound, provide a preoperative diagnosis, no real-time imaging methods exist for surgery to remove tumors altogether and assess surgical margins. Intraoperative pathology is not always available and can result in intraoperative delays and excessive costs. Frozen section analysis can add up to 30 minutes per case. Touch preparation increases the possibility of false-negative results and requires an experienced cytopathologist. An inaccurate SLNB can lead to misdiagnosis of the cancer stage. Consequently, there remains an unmet need for a real-time image-guided method for differentiating tumors from surrounding tissues during surgery and identifying positive lymph nodes during SLNB.

The present disclosure provides a method for medical imaging in a patient in need thereof, comprising administering a pharmaceutical composition comprising LS301 to the patient via slow intravenous injection at a dose between about 0.005 mg/kg and about 0.26 mg/kg.

The present disclosure further provides a method for surgical removal of cancer in a patient in need thereof, comprising administering a pharmaceutical composition comprising LS301 to the patient via slow intravenous injection over between about 30 seconds and about 4 minutes at a dose between about 0.001 mg/kg and about 0.1 mg/kg before surgery for cancer resection; imaging via near-infrared fluorescence of the LS301 to identify malignant tissue in the patient, wherein each patient serves as their own control in terms of the near-infrared (NIR) light on versus the NIR light off; and surgically removing the image-identified malignant tissue from the patient, wherein the imaging step occurs during the surgical removal step.

Other objects and features will be in part apparent and in part pointed out below.

### DETAILED DESCRIPTION

The present disclosure provides a method for medical imaging in a patient in need thereof, comprising administering a pharmaceutical composition comprising LS301 to the patient via slow intravenous injection at a dose between about 0.005 mg/kg and about 0.26 mg/kg.

In certain embodiments, the slow intravenous injection takes less than about 5 minutes to complete, for example, between about 30 seconds and about 5 minutes, between about 30 seconds and about 1 minute, between about 1 minute and about 1 minute 30 seconds, between about 1 minute 30 seconds and about 2 minutes, between about 2 minutes and about 2 minutes 30 seconds, between about 2 minutes 30 seconds and about 3 minutes, between about 3 minutes and about 3 minutes 30 seconds, between about 3 minutes 30 seconds and about 4 minutes, between about 4 minutes and about 4 minutes 30 seconds, or between about 4 minutes 30 seconds and about 5 minutes. In certain embodiments, the slow intravenous injection takes at least about 30 seconds to complete. In certain embodiments, the slow intravenous injection takes between about 30 seconds and 4 minutes to complete.

In certain embodiments, the rate of slow intravenous injection does not exceed about 2 mL/min. In certain embodiments, the rate of slow intravenous injection is between about 0.5 and about 2 mL/min, such as about 0.5 mL/min, about 0.6 mL/min, about 0.7 mL/min, about 0.8 mL/min, about 0.9 mL/min, about 1.0 mL/min, about 1.1 mL/min, about 1.2 mL/min, about 1.3 mL/min, about 1.4 mL/min, about 1.5 mL/min, about 1.6 mL/min, about 1.7 mL/min, about 1.8 mL/min, about 1.9 mL/min, or about 2 mL/min.

In certain embodiments, the dose is an initial dose of about 0.001 mg/kg (i.e., about 1 μg/kg). In certain embodiments, the dose is an initial dose of about 0.025 mg/kg. In certain embodiments, the initial dose is at least about 0.001 mg/kg. In certain embodiments, the initial dose is at least about 0.0025 mg/kg.

In certain embodiments, the dose ranges from about 0.001 mg/kg to about 0.025 mg/kg. In certain embodiments, the dose is chosen from 0.025 mg/kg, 0.0125 mg/kg, 0.00625 mg/kg, and 0.003125 mg/kg. In certain embodiments, the dose is about 0.025 mg/kg. In certain embodiments, the dose is about 0.0125 mg/kg. In certain embodiments, the dose is about 0.00625 mg/kg. In certain embodiments, the dose is about 0.003125 mg/kg.

In certain embodiments, the method further comprises uptitrating (or escalating) the initial dose to a target dose of between about 0.05 mg/kg and about 0.5 mg/kg. In various embodiments, the target dose is between about 0.001 mg/kg and about 0.025 mg/kg, between about 0.025 mg/kg and about 0.05 mg/kg, between about 0.05 mg/kg and about 0.075 mg/kg, between about 0.075 mg/kg and about 0.1 mg/kg, between about 0.1 mg/kg and about 0.2 mg/kg, between about 0.2 mg/kg and about 0.3 mg/kg, between about 0.3 mg/kg and about 0.4 mg/kg, and between about 0.4 mg/kg and about 0.5 mg/kg. In certain embodiments, the target dose is chosen from about 0.025 mg/kg, about 0.05 mg/kg, about 0.075 mg/kg, and about 0.1 mg/kg. In certain embodiments, the target dose is at least about 0.025 mg/kg. In certain embodiments, the target dose is less than about 0.1 mg/kg.

In certain embodiments, administration occurs before surgery for cancer resection. In certain embodiments, the administration occurs the day before surgery. In certain embodiments, the administration occurs about 1 to about 30 hours before the surgery, such as between about 1 hour and about 1.5 hours, between about 1.5 hours and about 2 hours, between about 2 hours and about 2.5 hours, between about 2.5 hours and about 3 hours, between about 3 hours and about 3.5 hours, between about 3.5 hours and about 4 hours, between about 4 hours and about 4.5 hours, between about 4.5 hours and about 5.0 hours, between about 5 hours and about 5.5 hours, between about 5.5 hours and about 6 hours, between about 6 hours and about 6.5 hours, between about 6.5 hours and about 7 hours, between about 7 hours and about 7.5 hours, between about 7.5 hours and about 8 hours, between about 8 hours and about 8.5 hours, between about 8.5 hours and about 9 hours, between about 9 hours and about 9.5 hours, between about 9.5 hours and about 10 hours, between about 10 hours and about 10.5 hours, between about 10.5 hours and about 11 hours, between about 11 hours and about 11.5 hours, between about 11.5 hours and about 12 hours, between about 12 hours and about 12.5 hours, between about 12.5 hours and about 13 hours, between about 13 hours and about 13.5 hours, between about 13.5 hours and about 14 hours, between about 14 hours and about 14.5 hours, between about 14.5 hours and about 15 hours, between about 15 hours and about 15.5 hours, between about 15.5 hours and about 16 hours, between about 16 hours and about 16.5 hours, between about 16.5 hours and about 17 hours, between about 17 hours and about 17.5 hours, between about 17.5 hours and about 18 hours, between about 18 hours and about 18.5 hours, between about 18.5 hours and about 19 hours, between about 19 hours and about 19.5 hours, between about 19.5 hours and about 20 hours, between about 20 hours and about 20.5 hours, between about 20.5 hours and about 21 hours, between about 21 hours and about 21.5 hours, between about 21.5 hours and about 22 hours, between about 22 hours and about 22.5 hours, between about 22.5 hours and about 23 hours, between about 23 hours and about 23.5 hours, between about 23.5 hours and about 24 hours, between about 24 hours and about 25 hours, between about 25 hours and about 26 hours, between about 26 hours and about 27 hours, between about 27 hours and about 28 hours, between about 28 hours and about 29 hours, or between about 29 hours and about 30 hours, before surgery. In certain embodiments, the administration occurs about 19 hours before surgery. In certain embodiments, the administration occurs between about 1.5 hours and about 4 hours before the surgery. In certain embodiments, the administration occurs about 1.5 hours to about 2.5 hours before the surgery. In certain embodiments, administration occurs at least about 1.5 hours before the surgery. In certain embodiments, the administration occurs less than about 4 hours before the surgery.

In certain embodiments, the administration occurs at least a day before surgery, such as 2 days, 3 days, 4 days, or 5 days before surgery. In certain embodiments, the administration occurs a day before surgery and 5 days before surgery. In certain embodiments, the administration occurs at least 2 days before surgery. In certain embodiments, the administration occurs not more than 2 days before surgery. In certain embodiments, the administration occurs at least 3 days before surgery. In certain embodiments, the administration occurs not more than 3 days before surgery. In certain embodiments, the administration occurs at least 4 days before surgery. In certain embodiments, the administration occurs not more than 4 days before surgery. In certain embodiments, the administration occurs at least 5 days before surgery. In certain embodiments, the administration occurs not more than 5 days before surgery.

In certain embodiments, administration occurs one day before surgery at a dose chosen from about 0.025 mg/kg, about 0.0125 mg/kg, about 0.00625 mg/kg, and about 0.003125 mg/kg.

In certain embodiments, the method further comprises imaging via near-infrared fluorescence of the LS301 to identify malignant tissue in the patient. In certain embodiments, the imaging is performed using an FDA-cleared imaging system capable of simultaneous fluorescence and white light imaging. In certain embodiments, the FDA-cleared imaging system has been cleared under § 510 (k), for example a device listed in Table 1.

TABLE 1
510(k)-cleared imaging system capable of simultaneous
fluorescence and white light imaging

| Device | Manufacturer |
|---|---|
| SPY Portable Handheld Imaging | Novadaq Technologies |
| (SPY-PHI) System | (now Stryker) |
| VS3-IR | Visionsense LTD (now Medronic) |
| [Note: | |
| now marketed as EleVision ™ in | |
| place of original VisionSense brand] | |
| Quest Spectrum ® (originally Artemis | Quest Medical Imaging |
| Handheld Fluorescence Imaging System) | |
| FLUOBEAM ® LX | Fluoptics |
| pde-neo II | Hamamatsu Photonics |
| VITOM ® NIR/ICG | Karl Storz |

In certain embodiments, the lymphatic system is mapped within a few minutes of IV delivery of LS301. In such embodiments, the same administration of LS301 identifies cancerous lymph nodes after the LS301 has cleared the lymphatics to allow detection of the lymph nodes, for example about 60 minutes after injecting the LS301 into the lymphatics.

In certain embodiments, LS301 is co-administered with Lymphoseek™ (⁹⁹Tc). In these embodiments, lymphatic mapping and identifying cancerous lymph nodes occur together.

In certain embodiments, the image identifies potentially cancerous tissue, including within a primary tumor site, other undiagnosed tumors, tumor margins, surgical cavity, or lymph nodes draining the primary tumor site in the patient. In certain embodiments, each patient serves as their own control in terms of near infrared (NIR) light on versus the NIR light off.

In certain embodiments, clinical data demonstrates that near infrared (NIR) imaging devices which excite with peak wavelengths between 800 nm to 810 nm and detect emission between 815 nm and 850 nm are suitable for use with LS301. Peak excitation and emission wavelengths will differ slightly when imaging in vivo, in situ, and ex vivo depending upon the matrix (e.g., tissue types, cells, and fluids).

In certain embodiments, LS301 is to be used with an NIR imaging system cleared by the FDA for specific use with LS301.

In certain embodiments, LS301 should be used by surgeons who have completed a training program on the use of NIR imaging systems for fluorescence imaging during surgery. In such cases, the device manufacturer typically provides the training.

In certain embodiments, LS301-IT is used with an FDA-cleared near infrared (NIR) imaging devices that has an acceptable emitting light source and ancillary excitation and emission filters to visualize fluorescence excitation in the wavelength of 800 nm to 810 nm and for observation from 815 nm to 850 nm. In such cases, the acceptable emitting light source comprises a laser or light-emitting diode (LED) with an appropriate power density expressed in mW/cm².

In certain embodiments, a tumor or SLN in the patient is identified before the excision of the tumor or SLN. In these embodiment, the surgeon is instructed, for example verbally or in writing via the professional labeling, to use the device and test for visible fluorescence through the patient's intact skin before making an incision. As a result, the LS301 locates the tumor or SLN before incision and may indicate one or more unexpected tumors or SLNs.

In certain embodiments, the method further comprises surgically removing the image-identified malignant tissue. In certain embodiments, the image-identified malignant tissue is selected from the group consisting of shave margins due to negative or positive tumor margins, residual malignant tissue in a surgical cavity, sentinel lymph node biopsy, and previously undiagnosed image-identified malignant breast or lymph node tissue.

In certain embodiments, the image-identified malignant tissue is shave margins due to negative tumor margins. In certain embodiments, the image-identified malignant tissue is shave margins due to positive tumor margins. In certain embodiments, the image-identified malignant tissue is residual malignant tissue in a surgical cavity. In certain embodiments, the image-identified malignant tissue is a sentinel lymph node biopsy. In certain embodiments, the image-identified malignant tissue is previously undiagnosed image-identified malignant breast tissue. In certain embodiments, the image-identified malignant tissue is previously undiagnosed image-identified malignant lymph node tissue.

In certain embodiments, the imaging step occurs during the surgical removal step. In these embodiments, the imaging step can occur before any incision is made to identify the location of a tumor or SLN through the patient's intact skin.

In certain embodiments, the patient has cancer and is chosen from ductal carcinoma in situ (DCIS) or Stage I-IV, primary invasive ductal carcinoma of a breast. In certain embodiments, the patient's primary surgical treatment options were partial mastectomy and, for those patients with invasive ductal carcinoma, simultaneous SLNB, with optional axillary lymph node dissection (ALND). In certain embodiments, the patients has DCIS and does not have SLNB. In certain embodiments, the patient has Stage I or II primary invasive ductal carcinoma of a breast. In certain embodiments, the patient has contralateral breast cancer. In certain embodiments, the patient has a second tumor of the same breast.

### Pharmaceutical Compositions

In certain embodiments, the pharmaceutical composition comprises 1-4 wt % a dye-peptide conjugate chosen from cypate-cyclo(Cys-Gly-Arg-Asp-Ser-Pro-Cys)-Lys-OH (LS301), or a salt thereof, wherein each amino acid residue is independently in a D or L configuration, 1-4 wt % calcium salt, 4-6 wt % polysorbate, 8-12 wt % beta-cyclodextrin, 0.2-2 wt % sodium acetate, 1-10 wt % dextrose, 1-100 mM histidine, and 62-86 wt % water.

In certain embodiments, the cypate is chosen from LS288, LS798, LS276, LS843, Cypate 2, Cypate 3, Cypate 4, Cy5, Cy5.5, Cy7, and Cy9. In certain embodiments, the cypate is Cy5.5. In certain embodiments, the cypate is Cy7. Cypates are known in the art. In many embodiments, the cypate is cypate 4.

In certain embodiments, the cypate is

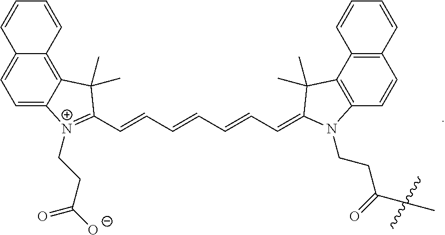 [SMILES: *C(=O)CCN1/C(=C/C=C/C=C/C=C/C2=[N+](CCC(=O)[O-])c3ccc4ccccc4c3C2(C)C)C(C)(C)c2c1ccc1ccccc21]

When depicted as a zwitterion, as in the structure above, a person of skill in the art would understand that the carboxylate anion will be protonated under the appropriate pH conditions and exist in equilibrium with the depicted anion.

In certain embodiments, at least one of the Cys amino acid residues is D-Cys.

In certain embodiments, the dye-peptide conjugate is LS301. In certain embodiments, the dye-peptide conjugate comprises the structure

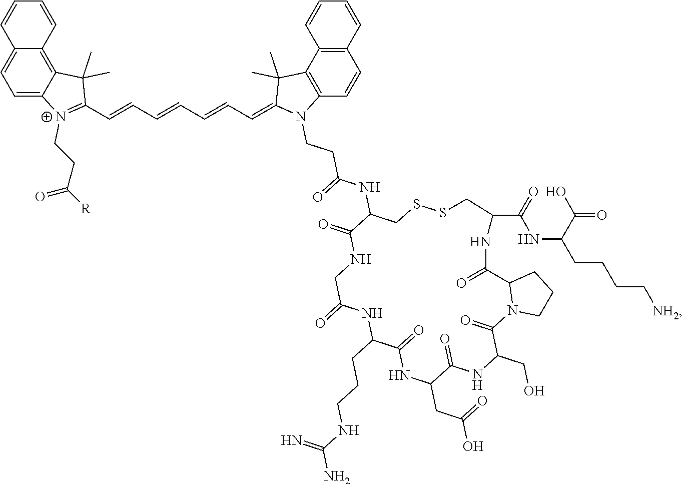

wherein R is OH or O.

In certain embodiments, the dye-peptide conjugate comprises the structure

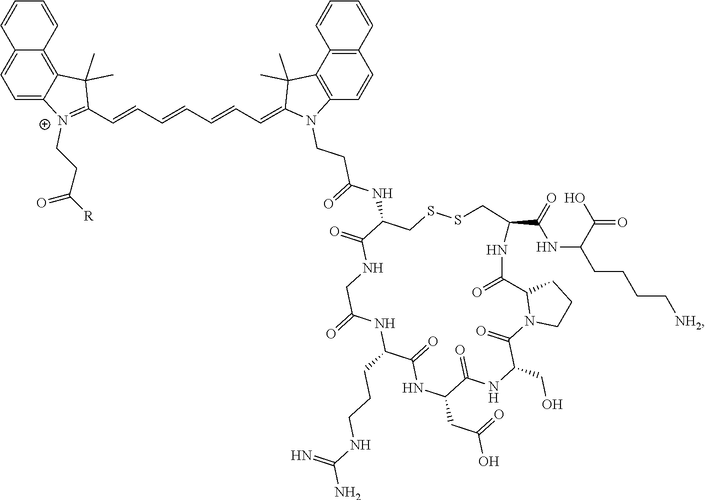

wherein R is OH or O.

In certain embodiments, the dye-peptide conjugate is LS301 and comprises the structural formula

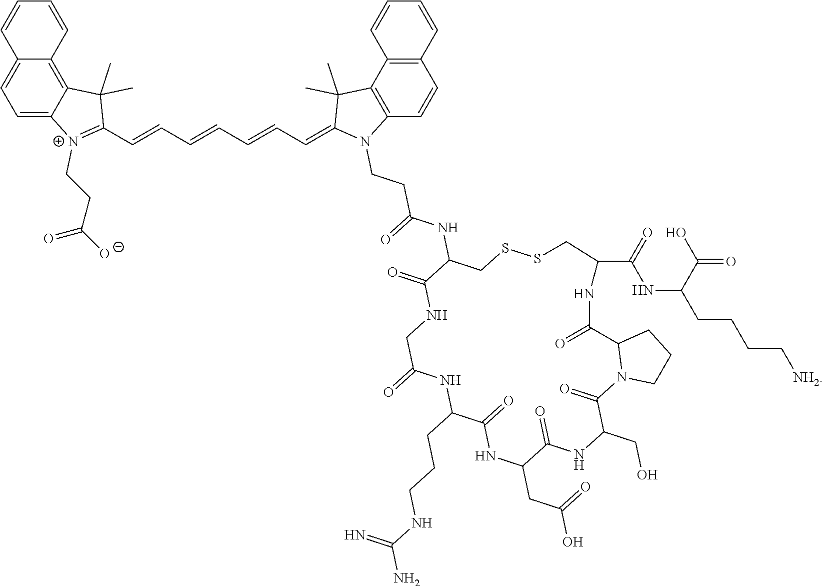 [SMILES: CC1(C)C(/C=C/C=C/C=C/C=C2/N(CCC(=O)NC3CSSCC(C(=O)NC(CCCCN)C(=O)O)NC(=O)C4CCCN4C(=O)C(CO)NC(=O)C(CC(=O)O)NC(=O)C(CCCNC(=N)N)NC(=O)CNC3=O)c3ccc4ccccc4c3C2(C)C)=[N+](CCC(=O)[O-])c2ccc3ccccc3c21]

In certain embodiments, the dye-peptide conjugate is LS301, comprising the structural formula

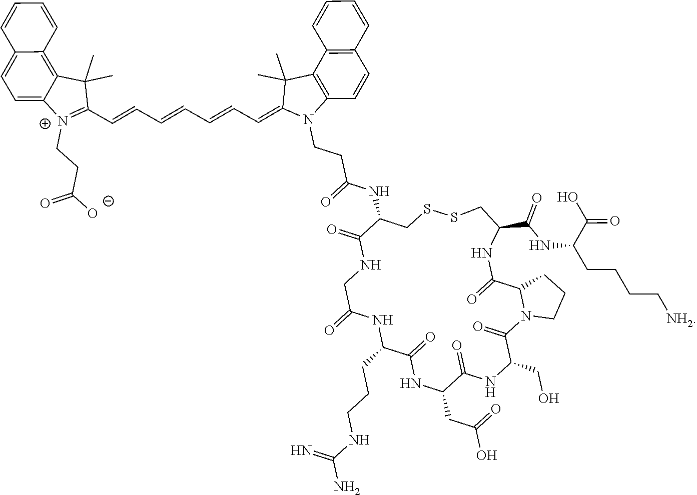

In certain embodiments, the pharmaceutical composition has been adjusted to a target pH of about 3.0 with acetic acid.

In certain embodiments, the pharmaceutical composition comprises about 1-4 wt % of LS301, such as about 1 wt %, about 2 wt %, about 3 wt %, or about 4 wt % dye LS301. In certain embodiments, the pharmaceutical composition comprises at least 1 wt % LS301.

In certain embodiments, LS838 may be substituted for LS301 in any of the lyophilized products or pharmaceutical compositions described herein.

In certain embodiments, the solution comprises between about 1 wt % and about 4 wt % calcium salt, such as about 1 wt %, about 2 wt %, about 3 wt %, or about 4 wt % calcium salt. In certain embodiments, the solution comprises at least 1 wt % calcium salt. In certain embodiments, the solution comprises not more than about 4 wt % calcium salt. In certain embodiments, the calcium salt for the solution is chosen from calcium gluconate, calcium citrate, calcium phosphate, calcium lactate, calcium lactate gluconate, calcium acetate, and calcium carbonate. In certain embodiments, the calcium salt in the solution is calcium gluconate.

In certain embodiments, the pharmaceutical composition comprises between about 4 wt % and about 6 wt % polysorbate, such as about 4 wt %, about 5 wt %, or about 6 wt % polysorbate. In certain embodiments, the pharmaceutical composition comprises at least 4 wt % polysorbate. In certain embodiments, the pharmaceutical composition comprises not more than 6 wt % polysorbate. In certain embodiments, the polysorbate for the pharmaceutical composition is chosen from polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate). In certain embodiments, the polysorbate for the pharmaceutical composition is polysorbate 80.

In certain embodiments, the pharmaceutical composition comprises between about 8 wt % and about 12 wt % beta-cyclodextrin, such as about 8 wt %, about 9 wt %, about 10 wt %, about 11 wt %, or about 12 wt % beta-cyclodextrin. In certain embodiments, the pharmaceutical composition comprises at least 8 wt % beta-cyclodextrin. In certain embodiments, the pharmaceutical composition comprises not more than about 12 wt % beta-cyclodextrin. In certain embodiments, the beta-cyclodextrin in the pharmaceutical composition is hydroxypropyl-beta-cyclodextrin.

In certain embodiments, the pharmaceutical composition comprises between about 0.2 wt % and about 2 wt % sodium acetate, such as between about 0.2 wt % and about 0.5 wt %, between about 0.5 wt % and about 1.0 wt %, between about 1.0 wt % and about 1.5 wt %, or between about 1.5 wt % and about 2.0 wt % sodium acetate. In certain embodiments, the pharmaceutical composition comprises at least about 0.2 wt % sodium acetate. In certain embodiments, the pharmaceutical composition comprises not more than about 2 wt % sodium acetate. In certain embodiments, the pharmaceutical composition comprises about 1 wt % sodium acetate. In certain embodiments, the sodium acetate is sodium acetate trihydrate.

In certain embodiments, the pharmaceutical composition comprises about 1-10 wt % dextrose, such as about 1 wt %, about 2 wt %, about 3 wt %, about 4 wt %, about 5 wt %, about 6 wt %, about 7 wt %, about 8 wt %, about 9 wt %, or about 10 wt % dextrose. In certain embodiments, the pharmaceutical composition comprises at least about 1 wt % dextrose. In certain embodiments, the pharmaceutical composition comprises not more than about 10 wt % dextrose. In certain embodiments, the pharmaceutical composition comprise about 4 wt % dextrose.

In certain embodiments, the pharmaceutical composition comprises about 0.5-2 wt % polysorbate, such as between about 0.5 wt % and about 1 wt %, between about 1 wt % and about 1.5 wt %, or between about 1.5 wt % and about 2 wt % polysorbate. In certain embodiments, the pharmaceutical composition comprises at least about 0.5 wt % pharmaceutical composition. In certain embodiments, the pharmaceutical composition comprises not more than about 2 wt % polysorbate. In certain embodiments, the pharmaceutical composition comprises about 1 wt % polysorbate. In certain embodiments, the polysorbate is polysorbate 80.

In certain embodiments, the pharmaceutical composition comprises about 1-100 mM histidine, such as between about 1 mM and about 10 mM, between about 10 mM and about 20 mM, between about 20 mM and about 30 mM, between about 30 mM and about 40 mM, between about 40 mM and about 50 mM, between about 50 mM and about 60 mM, between about 60 mM and about 70 mM, between about 70 mM and about 80 mM, between about 80 mM and about 90 mM, or between about 90 mM and about 100 mM histidine. In certain embodiments, the pharmaceutical composition comprises at least about 1 mM histidine. In certain embodiments, the pharmaceutical composition comprises not more than about 100 mM histidine. In certain embodiments, the pharmaceutical composition comprises about 10 mM histidine.

In various embodiments, the balance of the pharmaceutical composition is water, such as about 88-98.5 wt % water, for example, about 88 wt %, about 89 wt %, about 90 wt %, about 91 wt %, about 92 wt %, about 93 wt %, about 94 wt %, about 95 wt %, about 96 wt %, about 97 wt %, about 98 wt %, or about 98.5 wt % water. In certain embodiments, the pharmaceutical composition comprises at least about 88 wt % water. In certain embodiments, the pharmaceutical composition comprises not more than about 98.5 wt % water. In certain embodiments, the pharmaceutical composition comprises about 75 wt % water. In some embodiments, the water is water for injection.

In various embodiments, the balance of the pharmaceutical composition is water, such as 62-86 wt. % water, for example, about 62 wt. %, about 63 wt. %, about 64 wt. %, about 65 wt. %, about 66 wt. %, about 67 wt. %, about 68 wt. %, about 69 wt. %, about 70 wt. %, about 71 wt. %, about 72 wt. %, about 73 wt. %, about 74 wt. %, about 75 wt. %, about 76 wt. %, about 77 wt. %, about 78 wt. %, about 79 wt. %, about 80 wt. %, about 81 wt. %, about 82 wt. %, about 83 wt. %, about 84 wt. %, about 85 wt. %, or about 86 wt. % water. In certain embodiments, the pharmaceutical composition comprises at least about 62 wt. % water. In certain embodiments, the pharmaceutical composition comprises not more than about 86 wt. % water.

In certain embodiments, the pharmaceutical composition comprises about 2 wt % LS301, about 2 wt % calcium salt, about 5 wt % polysorbate, about 10 wt % beta-cyclodextrin, between about 1.2 and 1.4 wt % sodium acetate, about 4 wt % dextrose, about 10 mM histidine, and about 75 wt % water.

In certain embodiments, the pharmaceutical composition has a pH between about 5.5 and about 6.5.

In certain embodiments, the pharmaceutical composition has an osmolality of about 330 mOsm/kg.

The agents and compositions described herein can be formulated in any conventional manner using one or more pharmaceutically acceptable carriers or excipients. Such pharmaceutical compositions contain a therapeutically effective amount of a biologically active agent described herein, which can be in purified form, together with a suitable amount of carrier to provide the form for proper administration to the subject.

The pharmaceutical composition can be formulated to suit the mode of administration. The agents of use with the current disclosure can be formulated by known methods for administration to a subject using several routes, which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, rectal, and intra-lymphatic. The individual agents may also be administered with one or more additional agents or other biologically active or inert agents. Such biologically active or inert agents may be in fluid or mechanical communication with the agent(s) or attached to the agent(s) by ionic, covalent, Van der Waals, hydrophobic, hydrophilic, or other physical forces.

Agents or compositions described herein can also be used with other therapeutic modalities, as described further below. Thus, in addition to the therapies described herein, one may also provide to the subject other therapies known to be efficacious for treating the disease, disorder, or condition.

In certain embodiments, intraoperative imaging of LS301 during surgery assists the surgeon in distinguishing tumor tissue from noncancer tissue. In certain embodiments, the fluorescence image generated from LS-301 guides surgeons in the excision of tumor tissue margins and the biopsy of cancerous lymph nodes.

In certain embodiments, the dose of LS-301 is between about 0.16 and about 0.26 mg/kg. In certain embodiments, the dose of LS-301 in humans is about 0.006 mg/kg. In certain embodiments, the dose of LS-301 in humans is about 0.1 mg/kg. In certain embodiments, The 114 NOAEL is equivalent to a starting dose of about 0.025 mg/kg up to a target maximum dose of about 0.1 mg/kg. “NOAEL” refers to the “No-Observed-Adverse-Effect Level”. It is the highest tested dose or exposure level of a substance, such as LS301, at which no adverse effects are observed in a specific test population, usually animals or humans. The NOAEL is used to establish safe exposure levels for humans and to determine the dosage levels for the drug product.

The actual dosage amount of a compound of the present disclosure or composition comprising a compound of the present disclosure administered to a subject may be determined by physical and physiological factors such as type of animal treated, age, sex, body weight, the severity of the condition, the type of disease being treated, previous or concurrent therapeutic interventions, idiopathy of the subject and on the route of administration. A skilled artisan may determine these factors. The practitioner responsible for administration will typically determine the concentration of active ingredient(s) in a composition and the appropriate dose(s) for the individual subject. The individual physician may adjust the dosage in the event of any complication.

Agents and compositions described herein can be administered according to methods described herein in various means known to the art. The agents and composition can be used therapeutically as exogenous or endogenous materials. Exogenous agents are those produced or manufactured outside the body and administered to the body. Endogenous agents are those produced or manufactured inside the body by some device (biological or other) for delivery within or to other organs in the body.

### Definitions

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” indicates that a value includes the standard deviation of the mean for the device or method employed to determine the value. In some embodiments, the numerical parameters set forth in the written description and attached claims are approximations that can vary depending on the desired properties obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors resulting from the standard deviation found in their respective testing measurements. The recitation of ranges of values herein is merely intended to serve as a shorthand method of referring individually to each separate value falling within the range. Unless otherwise indicated herein, each value is incorporated into the specification as if it were individually recited herein. The recitation of discrete values is understood to include ranges between each value.

“Formulation” refers to preparing a drug in a form suitable for administration to a subject, such as a human. Thus, a “formulation” can include pharmaceutically acceptable excipients, including diluents or carriers. As used herein, “formulation” can refer to a lyophilized product or a pharmaceutical composition or a reconstitution diluent.

As used herein, “lyophilized product” refers to a product that has undergone freeze-drying, known as lyophilization, to remove water and preserve its structure and activity. In these embodiments, the lyophilized product is a dry and stable form of the drug, providing, for example, increased shelf-life and reduced storage costs.

A “stable” formulation or pharmaceutical composition or lyophilized product can refer to a composition having sufficient stability to allow storage at a convenient temperature, such as between about −20° C. and about 60° C., for a commercially reasonable time, such as at least about one day, at least about one week, at least about one month, at least about three months, at least about six months, at least about one year, or at least about two years.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” refers to a pharmaceutical carrier or excipient, it is implied that the carrier or excipient has met the required toxicological and manufacturing testing standards or is included in the Inactive Ingredient Guide prepared by the US Food and Drug Administration. “Pharmacologically active” (or “active”) as in a “pharmacologically active” (or “active”) derivative or analog refers to a derivative or analog having the same type of pharmacological activity as the parent compound and About equivalent in degree. The term “pharmaceutically acceptable salts” includes acid addition salts which are formed with inorganic acids such as, for example, hydrochloric or phosphoric acids, or such organic acids as acetic, oxalic, tartaric, mandelic, tosylic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

The term “pharmaceutically-acceptable carrier” is art-recognized. It refers to a pharmaceutically-acceptable material, composition, or vehicle, such as a liquid or solid filler, diluent, excipient, solvent, or encapsulating material, involved in carrying or transporting any subject composition or component thereof from one organ or portion of the body, to another organ, or portion of the body. Each carrier must be “acceptable” in compatibility with the subject composition and its components and not injurious to the patient. Some examples of materials that may serve as pharmaceutically acceptable carriers include sugars, such as lactose, glucose, and sucrose; starches, such as corn starch and potato starch; cellulose and its derivatives, such as sodium carboxymethyl cellulose, ethylcellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other nontoxic compatible substances employed in pharmaceutical compositions.

“Water for injection” or “WFI” refers to a type of purified water used for pharmaceutical applications, particularly in the preparation of drugs and medical devices. It is a highly purified form of water that meets the standards of the United States Pharmacopeia (USP), Chapter <1231>. WFI is substantially free of any substances that could potentially harm patients. WFI is produced via distillation or reverse osmosis, which removes impurities and other contaminants from the water, resulting in water substantially free of dissolved solids, organic matter, and microorganisms.

In some embodiments, “pharmaceutically acceptable salt” refers to acid addition salts with an inorganic or organic acid. Lists of suitable salts are found in WO 87/05297, Johnston et al., published Sep. 11, 1987; Remington's Pharmaceutical Sciences, 17^{th} ed., Mack Publishing Company, Easton, Pa., 1985, p. 1418; and J. Pharm. Sci., 66, 2 (1977), each incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002, which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts with an inorganic or an organic base. Inorganic bases which may be used to prepare salts include, for example, sodium, potassium, lithium, ammonium, calcium, magnesium, iron, zinc, manganese, aluminum hydroxides, carbonates, bicarbonates, phosphates, and the like; particularly preferred are the ammonium, potassium, sodium, calcium, and magnesium hydroxides, carbonates, bicarbonates, or phosphates. Organic bases from which may be used to prepare salts include, for example, primary, secondary, and tertiary amines, substituted amines including naturally occurring substituted amines, cyclic amines, basic ion exchange resins, and the like, specifically as isopropylamine, trimethylamine, diethylamine, triethylamine, tripropylamine, and ethanolamine.

As used herein, “medical imaging” or “medically imaging” refers to the use of various imaging technologies to produce visual representations of the internal or external structures and functions of a patient for diagnosis, treatment planning, or monitoring of medical conditions. Medical imaging includes, but is not limited to, X-ray, magnetic resonance imaging (MRI), computed tomography (CT), ultrasound, and fluorescence imaging. In certain embodiments, medical imaging refers to the use of fluorescence imaging to visualize and analyze the distribution and behavior of fluorescent molecules, such as LS301, in tissues and cells in a subject. In certain embodiments, medical imaging comprises detecting the presence of a signal emitted from a fluorescent molecule, such as LS301.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, “treating” or “treatment” refer to a subject at risk for developing a disease, or at risk of disease progression to a worse state. Prevention of a disease may involve complete protection from disease, for example as in the case of prevention of infection with a pathogen, or may involve prevention of disease progression, for example from prediabetes to diabetes. For example, prevention of a disease may not mean complete foreclosure of any effect related to the diseases at any level, but instead may mean prevention of the symptoms of a disease to a clinically significant or detectable level. Prevention of diseases may also mean prevention of the progression of a disease to a later stage of the disease.

The term “therapeutic agent” is art-recognized and refers to any chemical moiety that is a biologically, physiologically, or pharmacologically active substance acting locally or systemically in a subject. Examples of therapeutic agents, also referred to as “drugs,” are described in well-known literature references such as the Merck Index (14th edition), the Physicians' Desk Reference (64th edition), and The Pharmacological Basis of Therapeutics (12th edition). These therapeutic agents include, without limitation, medicaments; vitamins; mineral supplements; substances used for the treatment, prevention, diagnosis, cure, or mitigation of a disease or illness; substances that affect the structure or function of the body, or pro-drugs, which become biologically active or more active after they have been placed in a physiological environment.

“Weight percent” or “wt %” or “% (w/w)” refers to the ratio of the weight specified component in relation to the weight of the total composition, unless specified otherwise.

An “adverse event” is any untoward medical occurrence associated with treatment with a pharmaceutical composition described herein. A “mild adverse event” is easily tolerated by the subject, causes minimal discomfort, and does not interfere with everyday activities. A “moderate adverse event” is sufficiently discomforting to interfere with everyday activities; intervention may be needed. A “severe adverse event” prevents everyday activities; treatment or other intervention is usually needed. A “serious adverse event” results in death; is life-threatening (immediate risk of death from the event as it occurred); requires or prolongs inpatient hospitalization; results in persistent or significant disability/incapacity; or results in a congenital anomaly/disability, cancer, or drug overdose. An adverse event is incapacitating or disabling if it results in a substantial or permanent disruption of the subject's ability to conduct normal life functions.

A patient is said to “tolerate” a dose of a compound if administering that dose to that patient does not result in an unacceptable adverse event or an unacceptable combination of adverse events. One skill in the art will appreciate that tolerance is a subjective measure and that what may be tolerable to one patient may not be tolerable to a different patient. For example, one patient may not be able to tolerate a headache. In contrast, a second patient may find headaches tolerable but cannot tolerate vomiting. For a third patient, either headache alone or vomiting alone is tolerable, but the patient cannot tolerate the combination of headache and vomiting, even if the severity of each is less than when experienced alone.

“Maximum tolerated dose” means the highest drug dose or therapeutic dose that a patient can take without experiencing intolerable side effects. The maximum tolerated dose is typically determined empirically in clinical trials.

“Effective amount” and “therapeutically effective amount” of an agent, compound, drug, composition, or combination is an amount that is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the clinician's judgment.

“Informing” means referring to or providing published material, for example, providing an active agent with published material to a user, or presenting information orally, for example, by the presentation at a seminar, conference, or other educational presentation, by the conversation between a pharmaceutical sales representative and a medical care worker, or by the conversation between a medical care worker and a patient; or demonstrating the intended information to a user for comprehension.

“Medication Guide” means an FDA-approved patient labeling for a pharmaceutical product conforming to the specifications set forth in 21 CFR § 208 and other applicable regulations, which contains information for patients on how to safely use a pharmaceutical product. A medication guide is scientifically accurate and is based on, and does not conflict with, the approved professional labeling for the pharmaceutical product under 21 CFR § 201.57, but the language need not be identical to the sections of approved labeling to which it corresponds. For example, a medication guide with special risk management information is typically available for a pharmaceutical product.

“Patient package insert” means information for patients on how to safely use a pharmaceutical product that is part of the FDA-approved labeling. It is an extension of the professional labeling for a pharmaceutical product that may be distributed to a patient when dispensed, providing consumer-oriented information about the product in lay language. For example, it may describe benefits, risks, recognizing risks, dosage, or administration.

“Product” or “pharmaceutical product” means a dosage form of an active agent plus published material and, optionally, packaging.

“Product insert” means the professional labeling (prescribing information) for a pharmaceutical product, a patient package insert for the pharmaceutical product, or a medication guide for the pharmaceutical product.

“Professional labeling” or “prescribing information” means the official description of a pharmaceutical product approved by a regulatory agency (e.g., FDA or EMEA) regulating the marketing of the pharmaceutical product, which includes a summary of the essential scientific information needed for the safe and effective use of the drug, such for example indication and usage; dosage and administration; who should take it; adverse events (side effects); instructions for use in special populations (pregnant women, children, geriatric, etc.); safety information for the patient, and the like.

“Published material” means a medium providing information, including print, audio, visual, or electronic medium, for example, a flyer, an advertisement, a product insert, printed labeling, an internet website, an internet web page, an internet pop-up window, a radio or television broadcast, a compact disk, a DVD, an audio recording, or other recording or electronic medium.

“Risk” means the probability or chance of an adverse reaction, injury, or other undesirable outcome arising from medical treatment. An “acceptable risk” means measuring the risk of harm, injury, or disease arising from a medical treatment that an individual or group will tolerate. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At-risk” means a state or condition marked by a high level of risk or susceptibility. Risk assessment consists of identifying and characterizing the nature, frequency, and severity of the risks associated with using a product.

“Safety” means the incidence or severity of adverse events associated with the administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

“Up titration” or “escalating” of a compound refers to increasing the amount of a compound to achieve a therapeutic effect that occurs before dose-limiting intolerance for the patient. Up titration can be achieved in one or more dose increments, which may be the same or different.

“Down titration” or “deescalating” of a compound refers to decreasing the amount of a compound to balance the compounds therapeutic effect and dose-limiting intolerance for the patient. Like its counterpart, down titration can be achieved in one or more dose increments, which may be the same or different.

All methods described herein can be performed in any suitable order unless otherwise indicated herein or otherwise clearly contradicted by context. The use of all examples or exemplary language (e.g., “such as”) provided concerning certain embodiments herein is intended merely to better illuminate the present disclosure and does not limit the scope of the present disclosure otherwise claimed. No language in the specification should be construed as indicating any non-claimed element essential to the practice of the present disclosure.

Groupings of alternative elements or embodiments of the present disclosure are not construed as limitations. Each group member can be referred to and claimed individually or combined with other group members or elements found herein. One or more group members can be included or deleted from a group for convenience or patentability reasons. When any such inclusion or deletion occurs, the specification is herein deemed to contain the group as modified, thus fulfilling the written description of all Markush groups used in the appended claims.

All publications, patents, patent applications, and other references cited in this application are incorporated herein by reference in their entirety for all purposes to the same extent as if each publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### Enumerated Embodiments

Also disclosed herein are the following embodiments.

Embodiment 1: A method for medical imaging in a patient in need thereof, comprising administering a pharmaceutical composition comprising LS301 to the patient via intravenous delivery at a dose between about 0.001 mg/kg and about 0.5 mg/kg; and detecting the presence of a signal emitted from the LS301.

Embodiment 2: The method of Embodiment 1, wherein the pharmaceutical composition comprises 1~4 wt. % LS301, 1-4 wt. % calcium salt, 4-6 wt. % polysorbate, 8-12 wt. % beta-cyclodextrin, 0.2-2 wt. % sodium acetate, 0.2-10 wt. % dextrose, about 10 mM histidine, and 62-86 wt. % water.

Embodiment 3: The method of Embodiment 2, wherein the pharmaceutical composition comprises about 2 wt. % LS301, about 2 wt. % calcium salt, about 5 wt. % polysorbate, about 10 wt. % beta-cyclodextrin, about 1.3 wt. % sodium acetate, about 4 wt. % dextrose, about 10 mM histidine, and about 75 wt. % water.

Embodiment 4: The method of any one of Embodiments 1 to 3, wherein the intravenous delivery takes between about 30 seconds and about 4 minutes to complete.

Embodiment 5: The method of any one of Embodiments 1 to 4, wherein the intravenous delivery occurs at a rate of less than about 2 mL/min.

Embodiment 6: The method of any one of Embodiments 1 to 5, wherein the dose is chosen from about 0.025 mg/kg, about 0.0125 mg/kg, about 0.00625 mg/kg, and about 0.003125 mg/kg.

Embodiment 7: The method of any one of Embodiments 1 to 6, wherein the patient is in need of medical imaging for malignant tissue, potentially cancerous tissue, tumor margins, surgical cavity, a tumor site, or the site of drain of lymph nodes from a tumor site.

Embodiment 8: The method of any one of Embodiments 1 to 7, wherein the administration occurs before surgery for cancer resection.

Embodiment 9: The method of Embodiment 8, wherein the administration occurs between 1 day and 5 days before surgery

Embodiment 10: The method of Embodiment 8, wherein the administration occurs about 1.5 to about 4 hours before the surgery.

Embodiment 11: The method of Embodiment 10, wherein the administration occurs about 1.5 to about 2.5 hours before the surgery.

Embodiment 12: The method of any one of Embodiments 1 to 11, wherein the emitted signal is near-infrared fluorescence of the LS301, and malignant tissue is identified in the patient.

Embodiment 13: The method of Embodiment 11, wherein the medical imaging is performed using a 510 (k)-cleared imaging system capable of simultaneous fluorescence and white light imaging.

Embodiment 14: The method of Embodiment 10 or 11, wherein the image identifies potentially cancerous tissue, including within a primary tumor site, other undiagnosed tumors, tumor margins, surgical cavity, or lymph nodes draining the primary tumor site in the patient.

Embodiment 15: The method of any one of Embodiments 12 to 14, wherein each patient serves as their own control in terms of near-infrared (NIR) light on versus the NIR light off.

Embodiment 16: The method of any one of Embodiments 12 to 15, further comprising surgically removing the potentially cancerous tissue identified in the image.

Embodiment 17: The method of Embodiment 16, wherein the potentially cancerous tissue is selected from the group consisting of shave margins due to negative or positive tumor margins, residual malignant tissue in a surgical cavity, sentinel lymph node, and previously undiagnosed malignant breast or lymph node tissue.

Embodiment 18: The method of any one of Embodiments 16 or 17, wherein the imaging step occurs during the surgical removal step.

Embodiment 19: The method of any one of Embodiments 1 to 18, wherein the patient has cancer chosen from ductal carcinoma in situ (DCIS) or Stage I-IV, primary invasive ductal carcinoma of a breast.

Embodiment 20: The method of Embodiment 19, wherein the patient's primary surgical treatment options were partial mastectomy and simultaneous SLNB, with optional axillary lymph node dissection (ALND).

Embodiment 21: The method of any one of Embodiments 1 to 20, wherein the imaging is effective in terms of imaging results predicting the presence of tumor tissue confirmed by histopathology.

Embodiment 22: A method for surgical removal of cancer in a patient in need thereof, comprising administering or instructing administration of a pharmaceutical composition comprising LS301 to the patient via slow intravenous injection over between about 30 seconds and about 4 minutes at a dose between about 0.001 mg/kg and about 0.025 mg/kg between 1 day and 5 days before surgery for cancer resection; imaging via near-infrared fluorescence of the LS301 to identify malignant tissue in the patient, wherein each patient serves as their own control in terms of near-infrared (NIR) light on versus the NIR light off; and surgically removing the image-identified malignant tissue from the patient, wherein the imaging step occurs during the surgical removal step.

Embodiment 23: The method of Embodiment 22, wherein the pharmaceutical composition comprises 1~4 wt. % LS301, 1-4 wt. % calcium salt, 4-6 wt. % polysorbate, 8-12 wt. % beta-cyclodextrin, 0.2-2 wt. % sodium acetate, 0.2-10 wt. % dextrose, about 10 mM histidine, and 62-86 wt. % water.

Embodiment 24: The method of Embodiment 23, wherein the pharmaceutical composition comprises about 2 wt. % LS301, about 2 wt. % calcium salt, about 5 wt. % polysorbate, about 10 wt. % beta-cyclodextrin, about 1.3 wt. % sodium acetate, about 4 wt. % dextrose, about 10 mM histidine, and about 75 wt. % water.

Embodiment 25: The method of any one of Embodiments 22 to 24, wherein the dose is chosen from about 0.025 mg/kg, about 0.0125 mg/kg, about 0.00625 mg/kg, and about 0.003125 mg/kg.

Embodiment 26: The method of any one of Embodiments 22 to 25, wherein the imaging is performed using a 510 (k)-cleared imaging system capable of simultaneous fluorescence and white light imaging.

Embodiment 27: The method of any one of Embodiments 22 to 26, wherein the imaging identifies potentially cancerous tissue, including within primary tumor site, other undiagnosed tumors, tumor margins, surgical cavity, or lymph nodes draining the primary tumor site in the patient.

Embodiment 28: The method of Embodiment 27, wherein the image-identified malignant tissue is selected from the group consisting of shave margins due to negative or positive tumor margins, residual malignant tissue in the surgical cavity, sentinel lymph node biopsy, and previously undiagnosed image-identified malignant breast or lymph node tissue.

Embodiment 29: The method of any one of Embodiments 22 to 28, wherein the cancer is ductal carcinoma in situ (DCIS) or Stage I-II, primary invasive ductal carcinoma of a breast.

Embodiment 30: The method of Embodiment 29, wherein the patient's primary surgical treatment options were partial mastectomy and simultaneous SLNB, with optional axillary lymph node dissection (ALND).

### Examples

The following non-limiting examples are provided to further illustrate the present disclosure. It should be appreciated by those of skill in the art that the techniques disclosed in the examples that follow represent approaches the inventors have found function well in the practice of the present disclosure and thus can constitute examples of modes for its practice. However, those of skill in the art should, in light of the present disclosure, appreciate that many changes can be made in the specific embodiments disclosed and still obtain a like or similar result without departing from the spirit and scope of the present disclosure.

### Example 1—Lyophilized Product (LS301-IT)

A lyophilized product was formed by dissolving LS301 in a solution of calcium gluconate, polysorbate 80, hydroxypropyl-beta-cyclodextrin, and sodium acetate in water at a ratio of 1 gram of dye-peptide conjugate per 10 liters of the solution, filtering the LS301 solution, and lyophilizing the filtered LS301 solution to form the lyophilized product.

The amounts of each component in the lyophilized product are shown in Table 2. Table 3 compares the prior lyophilized product LS301-I (disclosed in copending U.S. patent application Ser. No. 17/119,305) and the lyophilized product LS301-IT shown in Table 2. Table 4 shows the drug product release specifications for LS301, including test items, test methods, and acceptance criteria.

TABLE 2
Composition of LS301 Lyophilized Product (LS301-IT)

| | | Amount per vial, | |
|---|---|---|---|
| Component | Function | mg/mL (wt %)^{a} | Quality Reference |
| LS301 drug substance | API | 2.0 | In-house |
| Calcium gluconate ^{b} | Stabilizer | 2.0 | USP, Ph Eur |
| Polysorbate 80 | Surfactant | 5 | Ph Eur, JP, NF |
| Hydroxypropyl- | Stabilizer | 10 | Ph Eur, JP, NF |
| beta-cyclodextrin | | | |
| Acetic acid, glacial | pH adjuster | Adjust to target pH 3.0 | USP-NF, Multi-Compendial |
| Sodium acetate | Buffer | 1.3 | Ph Eur, JP, NF |
| trihydrate | | | |
| Water for injection | Solvent | qs | USP |

API = active pharmaceutical ingredient; JP = Japanese Pharmacopoeia; NF = National Formulary; Ph Eur = European Pharmacopoeia; qs = quantum sufficit (sufficient quantity); USP = United States Pharmacopeia.
^{a}% w/w was calculated based on the density value of 1.0099 g/mL at 5° C.
^{b} Not listed in FDA's Inactive Ingredient Database. Approved as a prescription and over-the-counter (OTC) drug as a sterile injection at 100 mg/mL (10%) and in tablet dosage forms.

TABLE 3
Comparison of Pharmaceutical Compositions

| | LS301-HSA | | LS301-IT | |
|---|---|---|---|---|
| Dosage configuration | 2.0 | mg/vial | 6.0 | mg/vial |
| Lyophilized product | LS301 API: | | LS301 API: | |
| | 2.0 | mg/vial | 2.0 | mg/mL |
| | HSA: | | Calcium gluconate: | |
| | 100 | mg/vial | 2.0 | mg/mL |

| | WFI: qs | Polysorbate 80: | |
|---|---|---|---|
| | | 5 | mg/mL |
| | | Betadex (beta-cyclodextrin): | |
| | | 10 | mg/mL |
| | | Sodium acetate trihydrate: | |
| | | 1.28 | mg/mL |
| | | WFI: qs | |
| Reconstitution | Normal saline (0.9%) | 4% Dextrose (D-glucose), | |
| buffer | | 1% Polysorbate 80 in | |
| | | 10 | mM |
| | | Histidine | |
| pH after | ~6.7-7.0 | 5.5-6.5 | |
| reconstitution | | | |
| Osmolarity | Not tested | ~330 | mOsm/kg |

API = active pharmaceutical ingredient, HSA = human serum albumin; NA = not applicable; qs = quantum sufficit; WFI = water for injection.

TABLE 4
Drug Product Release Specification

| Test | Method | Acceptance Criteria |
|---|---|---|
| Appearance | Visual | Green to pale-green lyophilized cake or powder |
| | | Reconstitution diluent: Dark green colored solution, |
| | | free of visible particles. |
| Reconstitution time | USP <1> | Less than 2 minutes |
| ID A | HPLC or UPLC | The retention time of the main peak in the sample |
| | | corresponds to that of the standard. |
| ID B | HPLC or UPLC | The UV spectrum extracted at the main peak |
| | | maximum in the sample corresponds to that of the |
| | | standard |
| Constituted solution | USP <1>, | A: The solid dissolves completely, leaving no |
| | USP <790> | visible, undissolved matter. |
| | | B: The constituted solution is not significantly less |
| | | clear than an equal volume of the diluent in a |
| | | similar vessel and examined similarly. |
| | | C: The solution is essentially free from particles of |
| | | foreign matter that can be observed on visual |
| | | inspection. |
| Assay | HPLC or UPLC | 90.0-110.0% of label claim |
| Impurities | HPLC or UPLC | Any individual: NMT 5.0% |
| | | Total Report results |
| Content Uniformity | USP <905> | n = 10; L1 = 15.0 |
| (Weight Variation) | | n = 30; L1 = 15.0, L2 = 25.0 |
| Osmolality | USP <785> | Report result |
| pH | USP <791> | 5.5-6.5 |
| Particulates | USP <788> | NMT 6000 particles ≥ 10 μm per vial; |
| | | NMT 600 particles ≥ 25 μm per vial |
| Water determination | USP <921> | NMT 5.0% |
| (Karl Fisher) | | |
| Bacterial endotoxins | USP <85> | <4.2 EU/mg |
| Sterility | USP <71> | Meets the requirements of USP <71> for No growth |

EU = endotoxin unit; ID = identification; NMT = not more than; USP = United States Pharmacopeia; UV = ultraviolet; HPLC = high-pressure liquid chromatography; UPLC = ultrahigh-pressure liquid chromatography

From these results, the recommended long-term storage conditions for LS301-IT aILS301-HSA are −20° C. in a lyophilized powder form with protection from light. Under these conditions, both LS301 lyophilized products have been shown to be stable. However, many preclinical and nonclinical studies require the reconstituted pharmaceutical compositions to be prepared and maintained in solution for extended periods.

### Example 2—Reconstitution Diluent and Storage Stability

While developing LS301-IT, it was observed that reconstituting the lyophilized product with water for injection or saline did not maintain a pharmaceutical composition for the desired time of at least four hours. In particular, the LS301-HSA lyophilized product did not readily dissolve when introduced to saline or water for injection, but required sonication which increased the temperature and potentially the impurities after fully reconstituted. The aggregation and solubility issues were apparent visibly.

Therefore, a reconstitution diluent was developed to achieve a solution suitable for injection when stored for at least 8 hours at 25° C. and 24 hours when stored at 2-8° C. The diluent was manufactured by compounding, sterile filtration, and aseptic filling into vials, for example, containing at least 3 mL. The LS301-IT lyophilized product, when reconstituted with the diluent, did not present any issues for the reconstitution in become a dose ready for injection. The LS301-IT preparation (once reconstituted with reconstitution diluent) meets the acceptance criteria for injections as defined in the USP for particulate matter. The components of the reconstitution diluent are shown below in Table 5.

TABLE 5
Composition of Reconstitution Diluent

| | | Amount | |
|---|---|---|---|
| | | per vial, | |
| | | mg/mL | |
| Component | Function | (% w/w)^{a} | Quality Reference |
| Dextrose anhydrous | Tonicity agent | 40 (3.949%) | USP, Ph Eur, ACS |
| Polysorbate 80 | Surfactant | 10 (0.987%) | NF/Ph Eur/JP |
| L-histidine | Buffering agent | 1.5 (0.153%) | USP, Ph Eur, JP, ChP |
| Water for injection | Solvent | qs | USP, Ph Eur, JP |

ACS = American Chemical Society; ChP = Chinese Pharmacopoeia; JP = Japanese Pharmacopoeia; Ph Eur = European Pharmacopoeia; qs = quantum sufficit; USP = United States Pharmacopeia.

In certain embodiments, the lyophilized product is formulated with the solid ingredient of the diluent so that the lyophilized product can be reconstituted with water for injection, as shown in Table 6.

TABLE 6
LS301-IT formulated for reconstitution
with water for injection

| | Amount | wt % |
|---|---|---|
| | per vial, | of the |
| | mg/mL | lyophilized |
| Component | (% w/w)^{a} | product |
| LS301 drug substance | 2 | 2.8 |
| Calcium gluconate | 2 | 2.8 |
| Hydroxypropyl-beta- | 10 | 13.9 |
| cyclodextrin | | |
| Polysorbate 80 | 15 | 20.9 |
| Dextrose anhydrous | 40 | 55.7 |
| L-histidine | 1.5 | 2.1 |
| Sodium acetate trihydrate | 1.28 | 1.8 |
| Acetic acid, glacial | Adjust to target | |
| | pH 3.0 | |
| Total weight | 71.78 | 100.0 |

After reconstitution in the diluent, serial dilutions for LS301-IT (the lyophilized product of Table 2) and LS301-HSA were prepared from 2000 nmol to about 1 nmol. Replica sets of the LS3011 and the LS301-HSA dilutions were stored with light protection at −20° C., 4° C., and room temperature. Generally, room temperature is between about 20° C. and about 24° C.

Following reconstitution in diluent and at each time point before spectral analysis, each sample was inspected visually to assess sample homogeneity. Precipitation and flocculation were recorded.

For the spectroscopic study, the aliquots were also prepared in low-binding tubes before the solutions were transferred to a disposable cuvette (Brandtech Cat #759150) for steady-state measurements. All mixtures were vortexed for 30 seconds before the spectroscopy and kept in the dark except during analysis. The absorption and steady-state fluorescence spectra were recorded on a Beckman DU640 Spectrophotometer and a Horiba Fluorolog-3 spectrofluorometer. All measurements were conducted in triplicate at room temperature on Day 0 (preparation), Day 1, Day 3, and weekly through 6 weeks. Samples were excited at 760 nm to obtain most of the emission peak feature.

Both LS301 lyophilized products were stored at −20° C. and were thawed by placing them at room temperature for 10 minutes. No precipitate was observed in either sample. No noticeable precipitation or flocculation was observed for the LS301-IT dilutions stored at 4° C. or room temperature. The LS301-HSA samples stored at 4° C. and room temperature were stable through Week 1 as no flocculant was observed. From Week 2 through the end of the study (Week 6), some floc appeared in the solution. The floc increased throughout the study. However, after gentle swirling of the sample tubes, the floc redissolved.

The absorption spectrum of each sample was recorded before each fluorescence measurement. The peak absorbance of all the samples did not noticeably change during the 6-week testing period, indicating that the fluorescence loss of the samples was not from photobleaching but rather aggregation or dimerization.

The peak intensity of the fluorescence of each sample was recorded, calculated, and converted to a percent change compared to the fluorescence intensity of the freshly prepared sample (Day 0). A summary of the cumulated loss in fluorescence signal for each lyophilized product of LS301 at each of the three temperatures is shown in Table 7.

TABLE 7
Stability After Six Weeks of Storage.

| | Cumulated % Fluorescence Signal Loss | | |
|---|---|---|---|
| Lyophilized | Storage Temperature | | |
| product | −20° C. | 4° C. | RT |
| LS301-IT | 4.5% | 8.0% | 11.5% |
| LS301-HSA | 8.0% | 11.5% | 14.0% |

LS301-IT dilutions stored at −20° C. had the best stability as they lost only about 3.5% of the fluorescence signal over 6 weeks. The LS301-IT dilutions stored at 4° C. lost about 4.5% of the fluorescence signal, while the LS301-IT samples stored at RT lost ~8% of the fluorescence signal by Week 6. The resulting solution suitable for injection was stably stored for at least eight hours between 2° and 24° C. and 24 hours when stored between 2 and 8° C.

In this time frame, the LS301-HSA samples stored at −20° C. lost about 8% of the fluorescence over 6 weeks, the 4° C. samples lost about 11.5% of the fluorescence signal, and the room temperature samples lost about 14 of the fluorescence signal after 6 weeks. Thus, LS301-HSA was less stable than LS301-IT under these conditions.

All concentrations for each pharmaceutical composition followed the same pattern of lost fluorescence signal over 6 weeks. Thus, the lost fluorescence was independent of concentration.

Generally, fresh preparation of lyophilized LS301 drug product in diluent ensures the highest stability and fluorescence signal. However, based on these results, serial dilutions of either pharmaceutical composition can be prepared with the diluent, stored, and shipped frozen (e.g., −20° C.) with minimal loss of fluorescent signal. The LS301-IT lyophilized product is more stable than the LS301-HSA drug product when using the diluent for reconstitution, serial dilution, or long-term storage.

### Example 3—Mouse Study

Proof of concept imaging studies in mice showed that LS301 could bind and accumulate in tumors after a single IV delivery, producing a clear tumor-to-background ratio observed four hours post-injection. The biodistribution of LS301 showed high uptake in tumors. LS301 also accumulated in other tissues, especially the excretory organs of the liver and kidneys. LS301-IT showed excellent tumor uptake in 4T1 breast cancer tumor-bearing mice. Time-dependent noninvasive in-vivo fluorescence imaging of the LS301 in subcutaneous tumors using a Pearl small animal imager showed high fluorescence signal in LS301-IT compared to the surrounding tissue. Again a clear tumor-to-background ratio was observed four hours post-injection.

Results from safety pharmacology studies further support the potential safety of L301 and LS301-IT. No mortalities were observed in the in-vivo safety pharmacology studies in which male rats were treated with up to 20 mg/kg LS301. Apart from the transient LS301-related discoloration (green) noted in male rats following treatment with ≥10 mg/kg LS301, LS301 had no effects on functional observation battery (FOB) parameters, including motor activity, behavior changes, coordination, sensory/motor reflex responses, and body temperature. Similar urine discoloration was observed in the rat toxicity study in animals treated with ≥10 mg/kg LS301. The discoloration resolved on Day 15. LS301 did not affect cardiovascular function (IC₅₀ value greater than 9.981 μM) or respiratory system functional parameters (tidal volume, respiratory rate, and derived minute volume) up to 24 hours following a single IV administration of up to 20 mg/kg in rats. LS301 did not affect respiratory system functional parameters (tidal volume, respiratory rate, and derived minute volume) up to 24 hours following a single IV administration of up to 20 mg/kg LS301 in Sprague-Dawley rats. Results of the human Ether-à-go-go related gene (hERG) assay to evaluate LS301's potential to affect cardiovascular function showed an IC₅₀ value greater than 9.981 μM, suggesting LS301 is unlikely to affect cardiovascular function at the clinic. Rats tolerated LS301 well at doses up to 20 mg/kg, with no significant effects on central nervous, cardiovascular, and respiratory functions.

Appropriate bioanalytical methods were developed and validated to measure LS301 concentrations in rat plasma (LC-MS/MS) and various dosing formulations (HPLC-UV, UPLC) per good laboratory practice (GLP) regulations. The bioanalytical methods met acceptance criteria and were suitable for their intended use to determine the concentration of LS301 in pharmaceutical compositions or biological matrices.

The PK profile of LS301 in the rat was characterized by a half-life of fewer than five hours and systemic exposure that increased proportionally with dose. The volume of distribution appeared to be greater than the total volume of body water in humans, suggesting that LS301 is widely distributed to tissues following a single IV delivery. No differences were seen in the systemic exposure between male and female rats. LS301 exhibited high plasma protein binding, and no difference in plasma protein binding was observed among the species; mouse, rat, dog, minipig, and human. In vivo and ex vivo imaging experiments in a mouse breast cancer model also showed that LS301 distributes and accumulates in tumor and non-tumor tissues, especially the liver and kidneys, following a single IV delivery.

A single IV administration of LS301-IT at 1.0, 10, or 20 mg/kg LS301 to rats did not result in mortality. Instead, it resulted in non-adverse changes in serum chemistry and microscopic observations at all dose levels, urinalysis and clinical signs at ≤10 mg/kg, and macroscopic observations.

Test article-related clinical signs were limited to green discoloration of urine and green discoloration of skin at >10 mg/kg on Days 2 and 3 but were absent from Day 4 on. While some test article-related changes (discoloration of urine and skin, increased total serum bilirubin, decrease in kidney weight, a slight increase in adrenal glands, and increased prostate weights for males at 20 mg/kg) were observed in animals treated with ≥1 mg/kg LS301, these changes were absent by Day 15, were of low severity, and did not correlate with any associated degenerative or inflammatory changes in the kidney, adrenal glands or prostate gland. Therefore, the changes were considered non-adverse. Rats also tolerated LS301 and LS301-IT well at doses up to 20 mg/kg LS301. There were no mortalities or test article-related changes in body weight, food consumption, ophthalmology, hematology, coagulation, and urinalysis.

LS301 was negative for inducing structural/numerical chromosomal aberrations in Wolff-Bloom-Litton Chinese hamster ovary (CHO-WBL) cells with and without an S9 activation system. In a previous study, the potential for LS301 to cause genotoxicity was evaluated in a GLP bacterial mutagenicity (Ames) test in 4 strains of Salmonella typhimurium (TA100, TA1535, TA98, TA1537) and Escherichia coli (WP2uvrA) at doses up to 1000 μg/plate. The results were negative for LS301 mutagenicity.

In this study, the NOAEL was considered 20 mg/kg. The corresponding AUC₀₋₄₈ₕ and Co of LS301 at the established NOAEL on Day 1 were 14200 h*ng/ml and 69900 ng/mL for males and 14900 h*ng/mL and 98800 ng/mL for females, respectively. Therefore, the NOAEL of 20 mg/kg is equivalent to a 3.226 mg/kg human dose.

Based on the NOAEL in the rat toxicity study and results from the in-vivo safety pharmacology studies, a proposed starting dose of 0.025 mg/kg in the proposed clinical study (Example 4) would provide a safety margin of 129. Applying a correction factor of 0.88, the corrected NOAEL would be considered 17.6 mg/kg. A NOAEL of 17.6 mg/kg would provide a safety margin of 114 for the proposed clinical study with a starting dose of 0.025 mg/kg.

### Example 4—Phase 1b/2 Clinical Study of LS301-IT for Breast Cancer

This open-label, single-arm, Phase 1b/2 study investigates the safety, efficacy, and pharmacokinetics of LS301-IT in female patients undergoing a partial mastectomy and potentially a sentinel lymph node biopsy for breast cancer. LS301-IT is an optical imaging agent proposed to be indicated as an adjunct for intraoperative identification of malignant lesions in female patients undergoing a partial mastectomy and potentially a sentinel lymph node biopsy.

In this study, dose levels are 0.025, 0.05, 0.075, and 0.1 mg/kg, administered by slow intravenous (IV) injection (e.g., 3 mL of 0.1 mg/kg dose for 60 kg subject). In certain embodiments, the pharmaceutical composition is administered via IV delivery, such as an IV push or an IV injection. Administration over 5 minutes or more are listed as “IV push” or “IV infusion,” while administration of less than 5 minutes is listed as a “slow IV injection” or “slow IV push.” In certain embodiments, the slow intravenous administration does not exceed 2 mL per minute. Specific administration times may be provided for each patient.

The doses are based on preliminary findings from an earlier Phase 1 study using LS301-HSA and the nonclinical imaging study in a mouse breast cancer compared LS301-IT to the LS301-HSA pharmaceutical composition. A starting dose of 0.025 mg/kg is planned for the first cohort of patients in Phase 1b (Period 1) of the study. Patients in each dose cohort are monitored for safety, imaging, and PK of LS301-IT.

The LS301-IT clinical study commences as a Phase 1b (Period 1) using a modified toxicity probability interval (mTPI) study design, followed by a Phase 2a (Period 2) dose expansion and optimization study. Phase 2a is followed by a Phase 2b (Period 3) study, including the option to remove additional malignant tissue based on fluorescence.

The study design allows for expansion of the cohorts to assess fluorescence effectiveness while maintaining an acceptable safety profile. The starting dose of 0.025 mg/kg may not be the lowest dose in the planned dose range, as selection of the most effective dose is based on fluorescence imaging results and safety. A dose may be shown to be safe, but the fluorescence may be too intense for effective visualization, hence a lower doses may be tested. Period 1 is used to identify one or two low dose levels that are well-tolerated and provide sufficient fluorescence signal for the surgeon and a range of time intervals between dose administration and the start of surgery. Due to patient variability (e.g., PK, imaging at different timepoints, size of tumor, stage of disease, etc.), or two dose levels and a time intervals (e.g., 2-4 hours between administration and start of surgery) are assessed in an expanded patient population in Period 2.

Patients selected for the study have ductal carcinoma in situ (DCIS) or Stage I-II, primary invasive ductal carcinoma of the breast, for which the patient's primary surgical treatment is partial mastectomy and simultaneous SLNB (for patients with Stage I-II primary invasive ductal carcinoma), with or without axillary lymph node dissection (ALND). Axillary procedures may be performed before or after the primary lumpectomy at the surgeon's discretion. If the SLNB procedure is performed before lumpectomy, the mapping of sentinel lymph nodes (SLNs) should only be conducted using radiopharmaceuticals (e.g., Tc-99 sulfa colloid, Tc-99 tilmanocept). The optical imaging agent (dye) indocyanine green (ICG) cannot be used before the resection of the primary breast specimen and any additional shave margins.

In Period 1, up to three cohorts of three to six patients per cohort (i.e., a total of 18 patients) receive LS301-IT, starting within 2 (+0.5) hours before surgery. Dose administration may be up to four hours before surgery, depending on findings at two hours. This timing of LS301-IT injection relative to surgery supports both imaging and surgery on the same day, which is also more convenient for the patient.

Based on the safety and imaging results of the first cohort, doses of LS301-IT are selected for subsequent cohorts.

Imaging is performed in Period 1 using a 510 (k)-cleared imaging system capable of simultaneous fluorescence and white light imaging. The surgeon sees the fluorescence image on a color monitor superimposed onto or separate from the surgical field. The fluorescence image visualizes potentially cancerous tissue, including within the primary tumor site, other undiagnosed tumors, tumor margins, surgical cavity, or lymph nodes draining the primary tumor site in patients. The camera views the fluorescence image to aid detection and location of potentially malignant tissue both in situ and in ex-vivo specimens taken from the patient.

The LS301-IT dosing regimens identified in Period 1 as safe and potentially the most effective imaging results are investigated in larger cohorts of patients in the second period of the study. In Period 2 (Phase 2a study), the evaluation of LS301-IT for visualizing the malignant tissue is expanded to about 30 patients. In Period 3 (a Phase 2b study), the evaluation of LS301-IT for visualizing malignant tissue is expanded to about 50-60 patients, where the imaging results contribute to surgeon decisions on tissue removal. Eligible patients are enrolled in either Phase 1b (Period 1) or Phase 2a and b (Periods 2 and 3) of the study.

The primary objectives of the study are (1) to assess the safety of LS301-IT dosage and timing for the IV delivery before surgery and (2) to visualize the malignant tissue with LS301 based on binary assessments from the surgeon and/or pathologist, (a) to compare tumor to non-tumor ratio fluorescence response in different locations (e.g., primary tumor, lymph nodes if performed), (b) to evaluate the PK of LS301-IT, and (c) to evaluate the impact of adjustments in imaging techniques and conditions, including calibration and lighting, in distinguishing malignant tissue from nonmalignant tissue.

Phase 1b (Period 1) of the study is conducted first, and up to three cohorts of three to six patients per cohort receive LS301-IT by slow IV injection administered 2 (+0.5) hours before surgery. Depending on the results, the period before surgery may be increased to 4 hours. Target doses of LS301-IT are 0.025, 0.05, 0.075, and 0.1 mg/kg, with a planned starting dose (initial dose) of 0.025 mg/kg. The doses administered to subsequent cohorts of patients are based on recommendations after a review of the safety and imaging results.

Dosing regimens of LS301-IT identified in Period 1 as safe and potentially the most effective in terms of imaging results predicting the presence of tumor tissue confirmed by histopathology are expanded into larger cohorts of patients in Period 2 (a Phase 2a study) and Period 3 (a Phase 2b study). LS301-IT is further assessed to visualize malignant tissue in these larger cohorts. Additionally, in Period 3, the imaging results contribute to surgical decisions on tissue removal, the outcome of which (positive or negative for the tumor) is confirmed via histopathology results. Also, re-excision surgery may reduce the rate remission via LS301 use.

In each study period, each patient serves as their own control in terms of the near-infrared (NIR) light on versus the NIR light off. Replacement patients may be enrolled in either part if patients withdraw from the study before surgery/imaging. Patient participation in the study has a screening period (up to 28 days before the start of treatment). A treatment period consists of a single dose of LS301 IT administered by slow IV injection at 2 (±0.5) hours before the surgical procedure; depending on results, the period before surgery may be increased. The follow-up period is up to 35 days after surgery.

In Period 1, up to three cohorts of three to six patients per cohort receive LS301-IT (e.g., 3 mL) by slow IV injection at 2 (±0.5) hours before surgery. The IV administration time before surgery may be increased to four hours, depending on the results.

Doses of LS301-IT are 0.025, 0.05, 0.075, and 0.1 mg/kg. A 0.025 mg/kg starting dose (initial dose) is planned for the first cohort of patients. Patients in each cohort are monitored for safety and imaging. Based on the safety and imaging results of the first cohort, the dose, either dose escalation or dose de-escalation, of LS301-IT is selected for subsequent cohorts.

Based on the results of Period 1, Phase 2a (Period 2) and Phase 2b studies (Period 3) follow in which dosing regimens of LS301-IT identified in Period 1 as safe and potentially the most effective in terms of imaging results predictive of positive or negative tumor margins are investigated in larger cohorts of patients.

At least three cohorts of three to six patients per cohort (up to 18 patients in total in Period 1) receive LS301 IT. For each dose selected for each cohort, initially, three patients are tested. For doses observed to be most effective for safety and imaging, up to three additional patients per cohort are selected. That is six patients per cohort for the dosing cohort selected for Period 2. About 30 patients are enrolled in Period 2, and about 50 are enrolled in Period 3 to achieve sufficient evaluable patients. Eligible patients are enrolled in either Periods 1, 2, or 3 of the study. Replacement patients may be enrolled if patients withdraw from the study before surgery/imaging. Each patient serves as their own control in terms of NIR light on versus NIR light off.

Patients are included in the study if the following criteria apply:
Female patients are at least 18 years of age when signing informed consent.
DCIS or Stage I-II, primary invasive ductal carcinoma of the breast, for which the patient's primary surgical treatment is single breast partial mastectomy, and simultaneous SLNB is planned as part of the patient's therapy (for patients with Stage I-II primary invasive ductal carcinoma).
ECOG performance status of 0 to 2.
If of childbearing potential, the patient must have a negative serum pregnancy test and be using a medically acceptable form of contraception (e.g., hormonal birth control, double-barrier method) or abstinence.
Contraceptive use should be consistent with local regulations regarding the methods of contraception for those participating in clinical studies.

Patients are excluded from the study if any of the following criteria apply:
Contraindications for surgery.
In Period 1, simultaneous bilateral lumpectomies and bilateral partial mastectomies.
History of drug-related anaphylactic reactions, including those attributed to ICG or other agents used in the study.
Pulmonary disease, including uncontrolled airway hyperactivity, uncontrolled asthma, or asthma requiring oral corticosteroids.
Current diagnosis of any other active or clinically significant non-breast malignancy, which in the judgment of the Investigator, might interfere with LS301-IT imaging.
Total bilirubin level >1.5 ULN.
Aspartate aminotransferase (AST)/serum glutamic-oxaloacetic transaminase (SGOT) and alanine aminotransferase (ALT)/serum glutamic-pyruvic transaminase (SGPT) ≥2.5 times the upper limit of normal (ULN).
Prior neoadjuvant chemotherapy (Period 1 only), endocrine therapy, or biologic therapy for current clinically or biopsy-proven breast cancer.
Open surgery in the ipsilateral breast within one year before administration of LS301-IT
History of radiation therapy to the chest.
Received a systemic investigational drug of any kind for any indication within four weeks or five half-lives before administration of LS301-IT and/or the patient has received LS301-IT previously.
The patient is pregnant, breastfeeding, or plans to become pregnant within six months of administration of LS301-IT.

During Period 1, whether to proceed to the next cohort to be dosed is based on the safety criteria. The results from the previous cohort inform whether to proceed to the next cohort. During Periods 2 and 3, patients' safety is monitored, and dosing recommendations are made.

Patients are monitored for AEs from the time of LS301-IT injection through the End of Study Visit (Day 25 to 35) after surgery. AEs are graded according to event criteria per NCI CTCAE version 5.0 or higher. Action is taken according to the event's type and severity and whether the event meets the criteria of an SAE.

Depending on the severity of the event, new accruals to the dose may be temporarily held until the likely attribution or outcome of the AE is determined. Additionally, patients may choose to withdraw from the study at any time for any reason. A patient may be withdrawn from the study for any of the following reasons:
The patient is unwilling or unable to adhere to the protocol;
The patient develops symptoms or conditions listed in the exclusion criteria at any time during the study;
Any SAE, clinically significant AE, severe laboratory abnormality, intercurrent illness, or other medical condition that indicates to the Investigator that continued participation is not in the best interest of the patient; or
Other medical reasons.

Safety is assessed from AEs according to NCI-CTCAE (including SAEs and treatment discontinuation due to toxicity), clinical laboratory evaluations, vital signs, echocardiograms (ECGs), physical examinations, weight, concomitant medications, and Eastern Cooperative Oncology Group (ECOG) performance status.

Assessments of both the LS301-IT dose level and the time of LS301-IT injection to visualize the malignant tissue based on a binary scoring system of the intraoperative imaging and pathology data will be undertaken and obtained from the surgeon or pathologist. The tumor-to-non-tumor ratio fluorescence response is compared in different locations (e.g., primary tumor, lymph nodes, if performed). The impact of adjustments in imaging settings and conditions, including calibration and lighting, are also assessed in distinguishing malignant tissue from nonmalignant tissue.

During Periods 1 and 2, the effectiveness of LS301-IT in aiding intraoperative visualization of positive or negative tumor margins and malignant SLNs pre- and post-biopsy are assessed, and the accuracy of LS301-IT in correlating image-identified malignant breast or lymph node tissue with post-operative-pathology-findings are assessed.

During Period 3, the post-operative pathology findings are correlated with LS301-IT image-identified malignant tissue removed by the surgeon that would not otherwise have been removed, including shave margins due to negative or positive tumor margins, residual malignant tissue in the surgical cavity, SLNBs, and previously undiagnosed image-identified malignant breast or lymph node tissue.

During Periods 1 and 2, LS301-IT is assessed as an aid to intraoperative visualization of residual malignant tissue in the surgical cavity following resection and to detect additional tumors or potentially malignant lymph nodes during surgery not previously diagnosed, and the clinical value of LS301-IT for noninvasive pre-incision imaging use in the location of the primary tumor and draining lymph nodes. During Periods 2 and 3, a centralized review of sectioned specimens of the excised tumor and lymph node biopsies for staining phosphorylated ANXA2 is piloted.

Plasma PK of LS301-IT is assessed from blood samples collected from patients participating in Periods 1,2 and potentially 3. Blood samples may also be taken from a subset of patients in Period 2 to expand the PK population. Blood samples for PK analysis are collected before the LS301-IT injection and at regular intervals up to 24 hours post-injection. PK parameters such as maximum observed plasma concentration (Cₘₐₓ), time to maximum observed plasma concentration (Tₘₐₓ), the area under the plasma concentration time curve (AUC), terminal-elimination half-life (t½), the volume of distribution (Va), Clearance (CL), and bioavailability (F) would be collected.

Data are summarized descriptively. Table 8 summarizes the schedule of activities. About 100 patients are planned; up to 18 patients in Period 1, About 30 patients in Period 2, and About 50 patients in Period 3. The sample size is based on a common rationale for this type of Phase 1b/2 dose study and is not based on any statistical calculations.

TABLE 8
Schedule of Activities

| | | | | End of |
|---|---|---|---|---|
| | | Treatment | Interim | Study/Early |
| | Screening | Period | Visit | Termination |
| | 2-28 days | Day 1 | Day | Day |
| Activity | before surgery | (Baseline) ^{a} | 3 to 7 ^{b} | 25 to 35 ^{b} |
| Informed Consent | X | | | |
| Eligibility criteria, | X | | | |
| demographics, and medical | | | | |
| history (including prior cancer | | | | |
| therapies) ^{c} | | | | |
| Concomitant medications and | X | X | X | X |
| procedures | | | | |
| Physical examination ^{d} | X | X | X | X |
| Vital signs (including height | X | X | X | X |
| and weight) ^{e} | | | | |
| ECOG performance status ^{f} | X | X | | X |
| Electrocardiogram ^{g} | X | X | X | X |
| Hematology ^{h} | X | X | X | X |
| Clinical (blood) chemistry ⁱ | X | X | X | X |
| Coagulation ^{j} | X | | | |
| Urinalysis ^{k} | X | | | |
| Pregnancy test ^{l} | X | X | | X |
| Blood samples for PK ^{m} | | X | | |
| LS301-IT injection ⁿ | | X | | |
| Surgery ^{o} | | X | | |
| Imaging ^{p} | | X | | |
| Tissue Procurement/Pathology | | X | | |
| review ^{q} | | | | |
| Re-excision information | | | | X |
| (planned or actual) | | | | |
| Adverse events ^{r} | | X | X | X |

Abbreviations: AE = adverse event; ALP = alkaline phosphatase; ALT = alanine aminotransferase; aPTT = activated partial thromboplastin time; AST = aspartate aminotransferase; BUN = blood urea nitrogen; CrCl = creatinine clearance; CT = computed tomography; ECG = electrocardiogram; ECOG = Eastern Cooperative Oncology Group; EOS = end of the study; ET = early termination; hCG = human chorionic gonadotropin; INR = international normalized ratio; IV = intravenous; LDH = lactate dehydrogenase; MCH = mean corpuscular hemoglobin; MCV = mean corpuscular volume; ULN = upper limit of normal; WBC = white blood cell.
^{a} Day 1 is the Baseline of the study and the day of LS301-IT injection, followed by surgery for all patients.
b Post-surgical assessments will be performed between Days 3 and 7 and then again at EOS (Days 25 to 35) or ET.
^{c} Medical history includes confirmation of breast cancer diagnosis, significantconcomitant illnesses/diseases, prior therapy (for breast cancer and other prior cancer), andcurrent symptoms.
^{d} A complete physical examination will be performed at Screening or on Day 1 before LS301-IT injection. If a physical examination occurs during Screening, an abbreviated physical exam can be conducted on Day 1. Likewise, all subsequent physical examinations, i.e., between Day 3 and 7 and at EOS (Days 25 to 35) or ET, can be abbreviated.
^{e} Vital signs, including heart rate, blood pressure (supine), temperature, and respiratory rate, will be obtained at Screening, on Day 1 before LS301-IT injection, and at the end of LS301-IT and 1 hour (+15 minutes) after LS301 IT injection. Height (cm) should be measured at Screening only, but no later than on Day 1 before LS301-IT injection. Body weight will be collected at Screening, on Day 1 before LS301-IT injection, between Day 3 and 7, and at EOS (Days 25 to 35) or ET. Body weight collected on Day 1 will be used to calculate each patient's dose. Body weight should be measured within one day of the blood draw for clinical laboratory testing to determine calculated CrCl; weight and serum chemistry may be done the day before LS301-IT dosing at the discretion of the Investigator.
^{f} ECOG performance status will be assessed at Screening, on Day 1 before LS301-IT injection, and at EOS (Days 25 to 35) or ET. The Day 1 assessment may be done the day before LS301-IT injection at the discretion of the Investigator.
^{g} A 12-lead ECG will be obtained at Screening, on Day 1 at any time before LS301-IT injection on Day 1 within 60 minutes after the end of the LS301-IT injection, between Day 3 and 7, and at EOS (Days 25 to 35) or ET. The corrected QT (QTc Fridericia) is calculated based on the Screening ECG to assess subject eligibility (see Section 8.3.5). These data are entered into the eCRF. Interval data from subsequent ECGs is entered into the CRF.
^{h} Hematology includes RBC count, hematocrit, hemoglobin concentration, MCH, MCV, % reticulocytes, and WBC count with differential: basophils, eosinophils, lymphocytes, monocytes, neutrophils, and platelet count.
ⁱ Clinical chemistry includes ALP, ALT, AST, LDH; albumin, BUN, creatinine, glucose, total bilirubin, total protein, uric acid; bicarbonate, calcium, chloride, phosphorus, potassium, sodium; calculated CrCl is also required. Blood samples for clinical chemistry will be drawn at Screening, on Day 1 before LS301-IT injection, between Days 3 and 7, and at EOS (Days 25 to 35) or ET. All events of ALT ┌or AST┐ ≥3 × ULN and total bilirubin > 1.5 × ULN (>35% direct bilirubin) or ALT ┌or AST┐ ≥3 × ULN, and INR >1.5 (if INR measured), which may indicate severe liver injury (possible Hy's law), must be reported to [Sponsor] in an expedited manner.
^{j} Coagulation tests include aPTT, PT, and INR. Coagulation tests will be done on blood samples drawn at Screening.
^{k} Urinalysis with microscopic examination includes an assessment of blood, glucose, ketones, leukocytes, protein, pH, and specific gravity by dipstick. Microscopic examination (if blood or protein is abnormal). Urine will be inspected for appearance during collection (including color). Urinalysis will be done at the Screening.
^{l} A serum pregnancy test (hCG) should be done for all female patients of childbearing potential at Screening (central lab). A urine or serum pregnancy test should be done on Day 1 before LS301-IT injection (local lab), with results reviewed before LS301-IT injection to confirm patient eligibility. A urine or serum pregnancy test should be repeated at EOS (Days 25 to 35) or ET (central lab).
^{m} For patients participating in the PK blood sampling, times of blood sample collection for PK analysis are provided.
ⁿ It is planned that patients will receive a single dose of LS301-IT from a 2 mg/mL solution (e.g., 3 mL for a 0.1 mg/kg dose for a 60 kg patient) by slow IV injection (not exceeding 2 mL/min) at approximately 2 hours to as many as 4 hours before surgery on Day 1.
^{o} Surgery will be performed on Day 1 after an LS301-IT injection.
^{p} Fluorescence imaging will occur during the surgical procedure.
^{q} The surgeon will remove the tissue for pathology review per standard of care. Tissue and images may be sent to a Central Lab for additional review and analysis per the Exploratory Objective in Periods 2 & 3.
^{r} Patients will be assessed for AEs at the point LS301-IT is administered. Then, at about 15 minutes after administration of LS301-IT, patients will again be monitored for injection-related reactions.

Following the completion of Period 1 of the study, the LS301-IT dose level and the time of LS301-IT injection relative to surgery for Period 2 are selected. Following Period 2, the LS301-IT dose level and the time of LS301-IT injection relative to surgery for Period 3 are selected.

This study aims to produce improved contrast between malignant and healthy tissue, especially at the nodes and margins, so that the tissue can be surgically removed for analysis or as treatment. In some applications, a lower dose given a few days before surgery is expected to permit the dye-conjugate to bind to malignant tissue and flush from the other tissues so that background noise is minimized. At too low of a dose, not enough imaging agent is present to visualize the tissue of interest. At too high of a dose, the background signal overpowers the signal from the target tissue, thus preventing useful imaging.

### Example 5—Phase 1b/2 Clinical Study of LS301-IT for Breast Cancer

The aim of this Phase 1b/2 study is to investigate the safety, efficacy, and pharmacokinetics (PK) of a single dose of LS301-IT administered by intravenous (IV) injection in female patients undergoing partial mastectomy for DCIS (whether or not undergoing planned SLNB) or Stage I-II primary invasive breast cancer undergoing SLNB. Safety is the primary objective of this study, followed by efficacy, which will be assessed from fluorescence imaging observations and data.

This is a Phase 1b/Phase 2, open-label study to investigate the use of LS301-IT (investigational medicinal product [IMP]), a fluorescence imaging agent used for visualization of tumor margins and SLNs in female patients with DCIS or Stage I-II, primary invasive carcinoma of the breast, for which the patient's primary surgical treatment is partial mastectomy.

Eligible patients will be enrolled into either:
Phase 1b (Period 1): dose-finding (escalation/de-escalation), and dose timing adjustment;
Phase 2a (Period 2): expanded sample size based on acceptable dosing regimen, that being the dose level(s) and the time interval between LS301-IT injection and surgery, determined in Period 1; or
Period 2b (Period 3): allow surgeons to make additional surgical decisions based on fluorescence imaging findings during surgery
Period 3 will not be opened until results are available from Periods 1 and 2 and further FDA consultation is obtained).

The LS301-IT dosing amounts from this study are 0.025 mg/kg, 0.05 mg/kg, 0.075 mg/kg, and 0.1 mg/kg.

1. A method for medical imaging in a patient in need thereof, comprising:
administering a pharmaceutical composition comprising LS301 to the patient via intravenous delivery at a dose between about 0.001 mg/kg and about 0.5 mg/kg; and
detecting the presence of a signal emitted from the LS301.

2. The method of claim 1, wherein the pharmaceutical composition comprises:
1-4 wt. % LS301,
1-4 wt. % calcium salt,
4-6 wt. % polysorbate,
8-12 wt. % beta-cyclodextrin,
0.2-2 wt. % sodium acetate,
0.2-10 wt. % dextrose,
about 10 mM histidine, and
62-86 wt. % water.

3. (canceled)

4. The method of claim 1, wherein the intravenous delivery takes between about 30 seconds and about 4 minutes to complete.

5. The method of claim 1, wherein the intravenous delivery occurs at a rate of less than about 2 mL/min.

6. The method of claim 1, wherein the dose is chosen from about 0.025 mg/kg, about 0.0125 mg/kg, about 0.00625 mg/kg, and about 0.003125 mg/kg.

7. The method of claim 1, wherein the patient is in need of medical imaging for malignant tissue, potentially cancerous tissue, tumor margins, surgical cavity, a tumor site, or the site of drainage of lymph nodes from a tumor site.

8. The method of claim 1, wherein the administration occurs before surgery for cancer resection.

9-11. (canceled)

12. The method of claim 1, wherein the emitted signal is near-infrared fluorescence of the LS301, and malignant tissue is identified in the patient.

13. The method of claim 12, wherein the medical imaging is performed using a 510 (k)-cleared imaging system capable of simultaneous fluorescence and white light imaging.

14. The method of claim 12, wherein the image identifies potentially cancerous tissue, including within a primary tumor site, other undiagnosed tumors, tumor margins, surgical cavity, or lymph nodes draining the primary tumor site in the patient.

15. The method of claim 12, wherein each patient serves as their own control in terms of near-infrared (NIR) light on versus the NIR light off.

16-21. (canceled)

22. A method for surgical removal of cancer in a patient in need thereof, comprising:
administering or instructing administration of a pharmaceutical composition comprising LS301 to the patient via slow intravenous injection over between about 30 seconds and about 4 minutes at a dose between about 0.001 mg/kg and about 0.025 mg/kg between 1 day and 5 days before surgery for cancer resection;
imaging via near-infrared fluorescence of the LS301 to identify malignant tissue in the patient, wherein each patient serves as their own control in terms of near-infrared (NIR) light on versus the NIR light off; and
surgically removing the image-identified malignant tissue from the patient, wherein the imaging step occurs during the surgical removal step.

23. The method of claim 22, wherein the pharmaceutical composition comprises;
1-4 wt. % LS301,
1-4 wt. % calcium salt,
4-6 wt. % polysorbate,
8-12 wt. % beta-cyclodextrin,
0.2-2 wt. % sodium acetate,
0.2-10 wt. % dextrose,
about 10 mM histidine, and
62-86 wt. % water.

24. The method of claim 23, comprising:
about 2 wt. % LS301,
about 2 wt. % calcium salt,
about 5 wt. % polysorbate,
about 10 wt. % beta-cyclodextrin,
about 1.3 wt. % sodium acetate,
about 4 wt. % dextrose,
about 10 mM histidine, and
about 75 wt. % water.

25. The method of claim 22, wherein the dose is chosen from about 0.025 mg/kg, about 0.0125 mg/kg, about 0.00625 mg/kg, and about 0.003125 mg/kg.

26. The method of claim 22, wherein the imaging is performed using a 510 (k)-cleared imaging system capable of simultaneous fluorescence and white light imaging.

27. The method of claim 22, wherein the imaging identifies potentially cancerous tissue, including within primary tumor site, other undiagnosed tumors, tumor margins, surgical cavity, or lymph nodes draining the primary tumor site in the patient.

28. The method of claim 27, wherein the image-identified malignant tissue is selected from the group consisting of shave margins due to negative or positive tumor margins, residual malignant tissue in the surgical cavity, sentinel lymph node biopsy, and previously undiagnosed image-identified malignant breast or lymph node tissue.

29. The method of claim 22, wherein the cancer is ductal carcinoma in situ (DCIS) or Stage I-II, primary invasive ductal carcinoma of a breast.

30. The method of claim 29, wherein the patient's primary surgical treatment options were partial mastectomy and simultaneous SLNB, with optional axillary lymph node dissection (ALND).
